(19) United States
(12) Patent Application Publication
(10) Pub. No.: US 2018/0042808 A1
(43) Pub. Date: Feb. 15, 2018

(54) Vibrating, Essential Oils, Harmony Stone System, Apparatus and Method

(71) Applicant: Beth Hutchinson, West Charlton, NY (US)

(72) Inventor: Beth Hutchinson, West Charlton, NY (US)

(21) Appl. No.: 15/727,599

(22) Filed: Oct. 7, 2017

(51) Int. Cl. A61H 15/02; A61K 36/00; A61F 7/02; A61H 39/04; A61H 15/00; A61H 39/00

(52) U.S. Cl. CPC A61H 15/02; A61H 15/0092; A61H 39/007; A61F 7/02; A61H 39/04; A61K 36/00; A61H 2201/0165; A61H 2015/0007; A61H 2015/0042

(57) ABSTRACT

A therapeutic system and related method for use in massage, energy or other healing therapy, comprising: a therapeutic stone; a vibrator aperture carved into a top of the stone, passing only partially through the stone; at least one essential oil channel fabricated into a bottom of the vibrator aperture, passing the remainder of the way through from the bottom of the vibrator aperture to a bottom of the stone; a vibrator surrounded on a circumference thereof with silicon or equivalent material, sized to fit snugly but removably into the aperture; the aperture configured deeply enough in relation to a height of the vibrator to leave enough space for the aperture to provide a reservoir for essential oils poured into the aperture; the at least one essential oil channel configured for the essential oils to diffuse therethrough, through the bottom of the stone, and onto a body of a user.

### BACKGROUND OF THE INVENTION

Himalayan salt has been used in healing for thousands of years. Negative ions from the salt form when the stone is heated, which help clean the air and detoxify the body. The heat from the stone assists to melt fascia to help soothe muscles and joints. It helps detox the body and increase circulation while leaving the skin feeling refreshed. It can help ease, insomnia, blood, pressure, stress, anxiety and depression. Many massage therapists can incorporate this in their practice as well as energy workers. The stones can help ground and balance meridian levels, chakras and clear energy.

Essential oils have been around for thousands of years and are even mentioned in the bible. They can help aid in depression, anxiety and other illnesses. These oils can be applied topically, inhaled, or ingested. Many energy workers use this in their practice as oils can emit energy and enhance mediation when used on the different chakras.

### SUMMARY OF THE INVENTION

Disclosed herein is a therapeutic system and related method for use in massage, energy or other healing therapy, comprising: a therapeutic stone; a vibrator aperture carved into a top of the stone, passing only partially through the stone; at least one essential oil channel fabricated into a bottom of the vibrator aperture, passing the remainder of the way through from the bottom of the vibrator aperture to a bottom of the stone; a vibrator surrounded on a circumference thereof with silicon or equivalent material, sized to fit snugly but removably into the aperture; the aperture configured deeply enough in relation to a height of the vibrator to leave enough space for the aperture to provide a reservoir for essential oils poured into the aperture; the at least one essential oil channel configured for the essential oils to diffuse therethrough, through the bottom of the stone, and onto a body of a user.

### BRIEF DESCRIPTION OF THE DRAWINGS

The features of the invention believed to be novel are set forth in the appended claims. The invention, however, together with further objects and advantages thereof, may best be understood by reference to the following description taken in conjunction with the accompanying drawing(s) summarized below:

FIG. 1 illustrates top, side and bottom plan views of a Himalayan Salt or Soap Stone, modified to contain a vibrator receptacle/oil reservoir, and oil delivery channels.

FIG. 2 illustrates top, side and bottom plan views of a combination plug and vibrator, configured for secure insertion into the Stone of FIG. 1.

FIG. 3 is a top view photograph of a prototype of the Stone of FIG. 1, developed for experimental testing of the invention.

FIG. 4 is a top view photograph of a prototype of the Stone of FIG. 1, developed for experimental testing of the invention.

### DETAILED DESCRIPTION

The present invention combines in a novel and nonobvious manner, the natural elemental properties of a Himalayan salt stone or a Soap stone with the power of essential oils and the further power of healing vibration. The combination of all these elements operates in a synergistic fashion to increase circulation, reduce inflammation, improve sleep quality, promote relaxation, reduce tension and improve the user's sense of wellbeing. These benefits can heighten massage therapy sessions and/or energy work sessions.

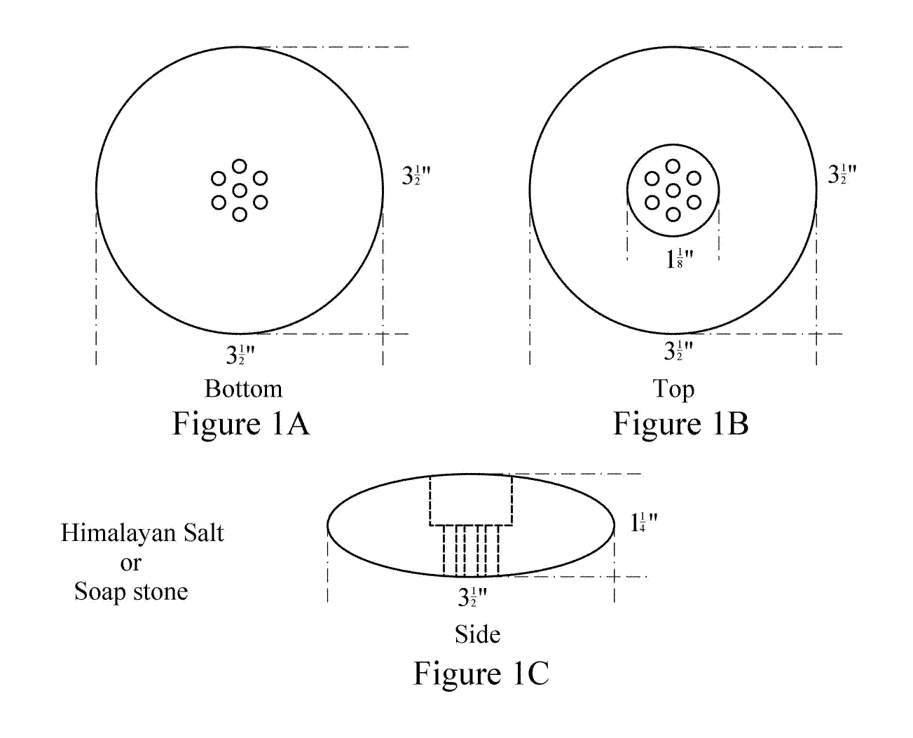

Specifically, FIG. 1 illustrates a Himalayan rock stone, with an inside section carved out to form a deep aperture, preferably but without limitation, with a circular cross section. At the bottom of the aperture are small holes forming oil delivery channels so when the stone is heated, the essential oil which is poured into the reservoir can pass through these holes and diffuse onto the body of the user.

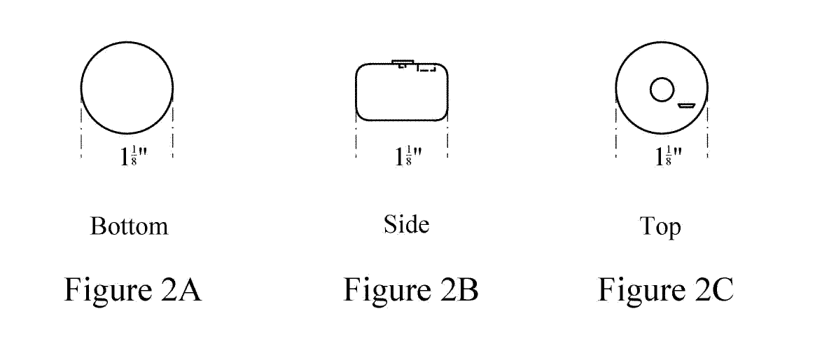

A vibration device illustrated in FIG. 2 is surrounded on a circumference thereof with silicon or equivalent material with similar frictional properties, and may have different selectable frequency and/or intensity settings. The silicon is fabricated to the shape of the aperture in the stone, and designed for a snug but removable fit. The stone aperture is deep enough in relation to the height of the vibration device to leave enough space for the oil reside after pouring, and to diffuse. The vibrator comprises a vibrating mechanism, battery, actuation button, and means for charging. The dimensions displayed in these two drawings are illustrative, not limiting.

To control the rate of oil flow through the oil delivery channels, these channels may contain cotton or equivalent porous wicking materials, so that the oils do not flow rapidly through the channels, but rather are disbursed over time.

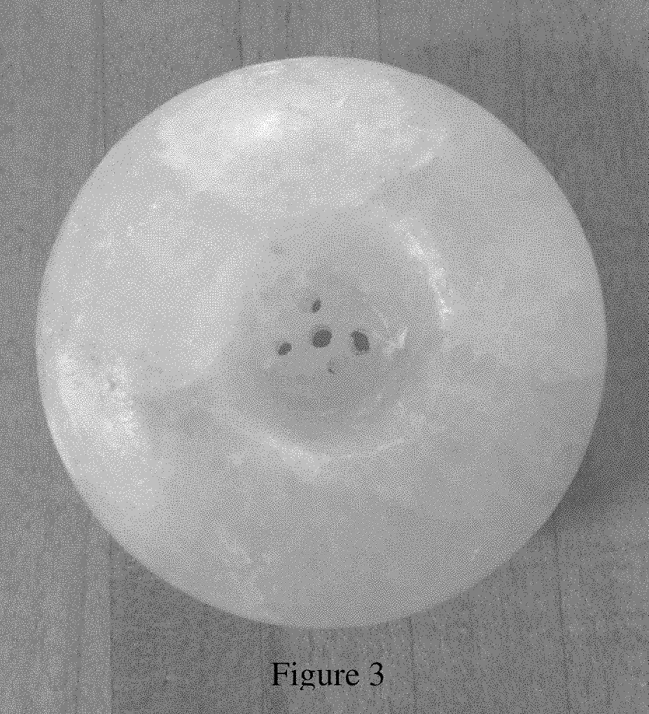

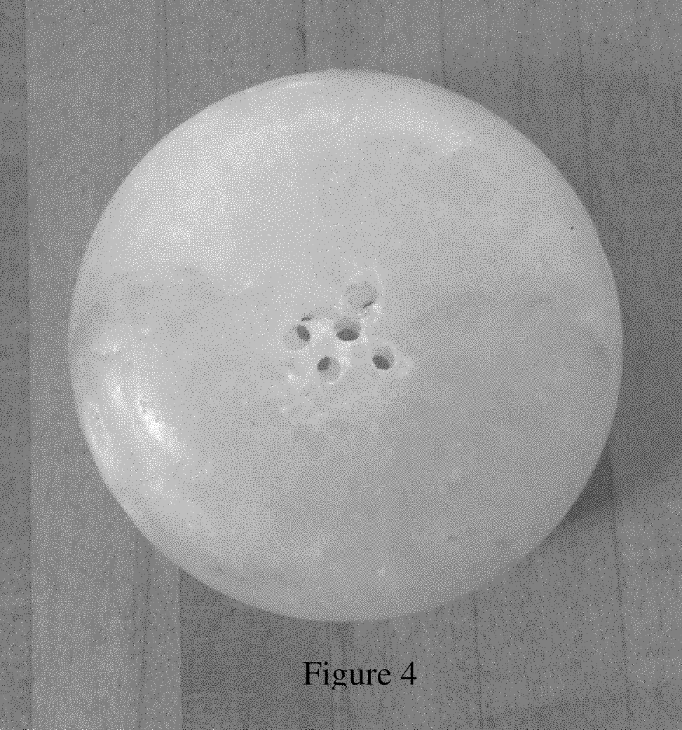

FIGS. 3 and 4 are photograph of a prototype of the Stone of FIG. 1, used for experimental testing of the invention.

The method of using the invention is to first pour the essential oils into the stone aperture. Then, the vibration device is inserted into the stone aperture to snug fit. The stone is then heated using either a microwave or a rice pack or stove cooker. Finally, the vibrator is turned on at the desired frequency and intensity setting, and the Stone is then used by the masseuse or energy worker on the user for therapy.

The knowledge possessed by someone of ordinary skill in the art at the time of this disclosure, including but not limited to the prior art disclosed with this application, is understood to be part and parcel of this disclosure and is implicitly incorporated by reference herein, even if in the interest of economy express statements about the specific knowledge understood to be possessed by someone of ordinary skill are omitted from this disclosure. While reference may be made in this disclosure to the invention comprising a combination of a plurality of elements, it is also understood that this invention is regarded to comprise combinations which omit or exclude one or more of such elements, even if this omission or exclusion of an element or elements is not expressly stated herein, unless it is expressly stated herein that an element is essential to applicant's combination and cannot be omitted. It is further understood that the related prior art may include elements from which this invention may be distinguished by negative claim limitations, even without any express statement of such negative limitations herein. It is to be understood, between the positive statements of applicant's invention expressly stated herein, and the prior art and knowledge of the prior art by those of ordinary skill which is incorporated herein even if not expressly reproduced here for reasons of economy, that any and all such negative claim limitations supported by the prior art are also considered to be within the scope of this disclosure and its associated claims, even absent any express statement herein about any particular negative claim limitations.

Finally, while only certain preferred features of the invention have been illustrated and described, many modifications, changes and substitutions will occur to those skilled in the art. It is, therefore, to be understood that the appended claims are intended to cover all such modifications and changes as fall within the true spirit of the invention.

I claim:

1. A therapeutic system for use in massage, energy or other healing therapy, comprising:
a therapeutic stone;
a vibrator aperture carved into a top of said stone, passing only partially through said stone;
at least one essential oil channel fabricated into a bottom of said vibrator aperture, passing the remainder of the way through from said bottom of said vibrator aperture to a bottom of said stone;
a vibrator surrounded on a circumference thereof with silicon or equivalent material, sized to fit snugly but removably into said aperture;
said aperture configured deeply enough in relation to a height of said vibrator to leave enough space for said aperture to provide a reservoir for essential oils poured into said aperture;
said at least one essential oil channel configured for the essential oils to diffuse therethrough, through said bottom of said stone, and onto a body of a user of said system.

2. The system of claim 1, said therapeutic stone comprising Himalayan Salt.

3. The system of claim 1, said therapeutic stone comprising Soap Stone.

4. The system of claim 1, said vibrator comprising at least two different selectable frequencies.

5. The system of claim 1, said vibrator comprising at least two different selectable intensities.

6. The system of claim 1, said at least one essential oil channel comprising a porous material for controlling a flow rate of the oils through said at least one essential oil channel.

7. A method of providing massage, energy or other healing therapy, comprising:
providing a therapeutic stone with a vibrator aperture carved into a top of said stone and passing only partially through said stone, and with at least one essential oil channel fabricated into a bottom of said vibrator aperture passing the remainder of the way through said stone from said bottom of said vibrator aperture to a bottom of said stone;
providing a vibrator surrounded on a circumference thereof with silicon or equivalent material, sized to fit snugly but removably into said vibrator aperture;
pouring essential oils into said aperture, which aperture is also configured deeply enough in relation to a height of said vibrator to leave enough space for said aperture to provide a reservoir for said essential oils;
inserting said vibrator into said aperture;
heating said stone;
turning on said vibrator;
placing said heated, vibrating stone containing essential oils onto a body of a user of said system;
diffusing said essential oils onto the user's body through said at least one essential oil channel; and
thereafter, removing said vibrator from said aperture.

8. The method of claim 7, said therapeutic stone comprising Himalayan Salt.

9. The method of claim 7, said therapeutic stone comprising Soap Stone.

10. The method of claim 7, further comprising selecting a vibrator frequency from at least two different selectable frequencies.

11. The method of claim 7, further comprising selecting a vibrator frequency from at least two different selectable intensities.

12. The method of claim 7, further comprising controlling a flow rate of the oils through said at least one essential oil channel using a porous material as part of said at least one essential oil channel.
